(19) United States
(12) Patent Application Publication
(10) Pub. No.: US 2008/0154314 A1
(43) Pub. Date: Jun. 26, 2008

(54) Composite interference screw for attaching a graft ligament to a bone, and other apparatus for making attachments to bone

(76) Inventor: Dennis M. McDevitt, Raleigh, NC (US)

Correspondence Address: Mark J. Pandiscio;Pandiscio & Pandiscio, P. C., 470 Totten Pond Road, Waltham, MA, 02451-1914, US

(21) Appl. No.: 11/893,440

(22) Filed: Aug. 16, 2007

Related U.S. Application Data
(60) Provisional application No. 60/838,119, filed on Aug. 16, 2006.

(51) Int. Cl. A61B 17/56; A61F 2/08

(52) U.S. Cl. 606/304; 623/13.14

(57) ABSTRACT

An interference screw comprising:
an open helical coil having a proximal end and a distal end aligned along a longitudinal axis and defining an internal volume, with the internal volume communicating with the region exterior to the open helical coil through the spacing between the turns of the open helical coil; and
at least one runner disposed within the internal volume and connected to multiple turns of the open helical coil.

### REFERENCE TO PENDING PRIOR PATENT APPLICATION

This patent application claims benefit of pending prior U.S. Provisional Patent Application Ser. No. 60/838,119, filed Aug. 16, 2006 by Dennis M. McDevitt for COMPOSITE INTERFERENCE SCREW FOR ATTACHING A GRAFT LIGAMENT TO A BONE, AND OTHER APPARATUS FOR MAKING ATTACHMENTS TO BONE (Attorney's Docket No. INCUMED-02 PROV), which patent application is hereby incorporated herein by reference.

### FIELD OF THE INVENTION

This invention relates to medical apparatus and procedures in general, and more particularly to medical apparatus and procedures for reconstructing a ligament and/or making attachments to bone.

### BACKGROUND OF THE INVENTION

Ligaments are tough bands of tissue which serve to connect the articular extremities of bones, or to support and/or retain organs in place within the body. Ligaments are typically made up of coarse bundles of dense fibrous tissue which are disposed in a parallel or closely interlaced manner, with the fibrous tissue being pliant and flexible but not significantly extensible.

In many cases, ligaments are torn or ruptured as the result of an accident. As a result, various procedures have been developed to repair or replace such damaged ligaments.

For example, in the human knee, the anterior and posterior cruciate ligaments (i.e., the “ACL” and “PCL”) extend between the top end of the tibia and the bottom end of the femur. The ACL and PCL serve, together with other ligaments and soft tissue, to provide both static and dynamic stability to the knee. Often, the anterior cruciate ligament (i.e., the ACL) is ruptured or torn as the result of, for example, a sports-related injury. Consequently, various surgical procedures have been developed for reconstructing the ACL so as to restore substantially normal function to the knee.

In many instances, the ACL may be reconstructed by replacing the ruptured ACL with a graft ligament. More particularly, in such a procedure, bone tunnels are generally formed in both the top of the tibia and the bottom of the femur, with one end of the graft ligament being positioned in the femoral tunnel and the other end of the graft ligament being positioned in the tibial tunnel. The two ends of the graft ligament are anchored in place in various ways well known in the art so that the graft ligament extends between the bottom end of the femur and the top end of the tibia in substantially the same way, and with substantially the same function, as the original ACL. This graft ligament then cooperates with the surrounding anatomical structures so as to restore substantially normal function to the knee.

In some circumstances, the graft ligament may be a ligament or tendon which is harvested from elsewhere in the patient's body (e.g., a semitendinosus tendon and/or a gracilis tendon); in other circumstances, the graft ligament may be harvested from a cadaver; and in still other circumstances, the graft ligament may be a synthetic device. For the purposes of the present invention, all of the foregoing may be collectively referred to herein as a “graft ligament”.

As noted above, various approaches are well known in the art for anchoring the graft ligament in the femoral and tibial bone tunnels.

In one well-known procedure, which may be applied to femoral fixation, tibial fixation, or both, the end of the graft ligament is placed in the bone tunnel, and then the graft ligament is fixed in place using a headless orthopedic screw, generally known as an “interference” screw. More particularly, with this approach, the end of the graft ligament is placed in the bone tunnel and then the interference screw is advanced into the bone tunnel so that the interference screw extends parallel to the bone tunnel and simultaneously engages both the graft ligament and the side wall of the bone tunnel. In this arrangement, the interference screw essentially drives the graft ligament laterally, into engagement with the opposing side wall of the bone tunnel, whereby to secure the graft ligament to the host bone with a so-called “interference fit”.

See, for example, FIGS. 1 and 2, where a graft ligament 5 is secured to a host bone 10 by an interference screw 15. More specifically, graft ligament 5 (e.g., a doubled-over semitendinosus tendon whip-stitched together at one end) is disposed in a bone tunnel 20 (e.g., by towing it up into the bone tunnel 20 with a tow suture 25). Then interference screw 15 is advanced into position between graft ligament 5 and side wall 20A of bone tunnel 20, so as to drive graft ligament 5 against the opposite side wall 20B of bone tunnel 20, whereby to bind graft ligament 5 in bone tunnel 20 (and hence to host bone 10) with an interference fit. Thereafter, over time (e.g., several months), the graft ligament and the host bone grow together at their points of contact so as to provide a strong, natural joinder between the ligament and the bone.

Interference screws have proven to be an effective means for securing a graft ligament in a bone tunnel. However, the interference screw itself generally takes up a substantial amount of space within the bone tunnel, which can limit the surface area contact established between the graft ligament and the side wall of the bone tunnel. This in turn limits the region of bone-to-ligament ingrowth, and hence can affect the strength of the joinder.

For this reason, substantial efforts have been made to provide interference screws fabricated from absorbable materials, so that the interference screw can eventually disappear and bone-to-ligament ingrowth can take place about the entire perimeter of the bone tunnel. To this end, various absorbable interference screws have been developed which are made from biocompatible, bioabsorbable polymers, e.g., polylactic acid (PLA), polyglycolic acid (PGA), etc. These polymers generally provide the substantial mechanical strength needed to set the interference screw into position and to hold the graft ligament in position while bone-to-ligament ingrowth occurs, without remaining in position on a permanent basis.

In general, interference screws made from such biocompatible, bioabsorbable polymers have proven successful. However, these absorbable interference screws still suffer from several disadvantages. First, clinical evidence suggests that the quality of the bone-to-ligament ingrowth is somewhat different than natural bone-to-ligament ingrowth, in the sense that the aforementioned bioabsorbable polymers tend to be replaced by a fibrous mass rather than a well-ordered tissue matrix. Second, clinical evidence suggests that absorption is sometimes incomplete, leaving a substantial foreign mass remaining within the body. This problem is exacerbated by the fact that interference screws tend to be fairly large, e.g., it is common for an interference screw to have a diameter (i.e., an outer diameter) of 8-12 mm and a length of 20-25 mm.

Bone scaffold structures have been developed which provide a temporary scaffold to support bone growth and which are then substantially completely replaced by the new bone. Thus, these bone scaffold structures can provide superior bone-to-ligament ingrowth and a more complete absorption. The bone scaffold structures may be formed out of a synthetic material (e.g., resorbable polymers), an allograft material (e.g., demineralized bone) and/or other materials (e.g., hydroxyapatite). Furthermore, the bone scaffold structures may be “doped” with bone growth factors so as to enhance bone ingrowth. However, these bone scaffold structures are relatively weak and brittle, and hence are not good candidates for forming interference screws, i.e., these bone scaffold structures lack the short term mechanical strength needed to set the interference screw into position and to hold the graft ligament in position while bone-to-ligament ingrowth occurs.

Thus, there is a need for a new interference screw which (i) has the short term strength needed to set the interference screw into position and to hold the graft ligament in position while bone-to-ligament ingrowth occurs, (ii) promotes superior bone-to-ligament ingrowth, and (iii) substantially completely disappears from the surgical site over time.

There are also many other situations in which attachments need to be made to bone. In many of these situations, it would be advantageous to have new apparatus for making attachments to bone which (i) has the short term strength needed to set the apparatus into position and to hold the various elements in position while bone ingrowth occurs, (ii) promotes superior bone ingrowth, and (iii) substantially completely disappears from the surgical site over time.

### SUMMARY OF THE INVENTION

These and other objects are addressed by the provision and use of novel apparatus for making attachments to bone.

In one preferred form of the invention, there is provided a novel composite interference screw for attaching a graft ligament to a bone. The composite interference screw comprises a screw frame for providing the short term strength needed to set the interference screw into position and to hold the graft ligament in position while bone-to-ligament ingrowth occurs, and an ingrowth core for promoting superior bone-to-ligament ingrowth. Preferably, the screw frame comprises a bioabsorbable polymer, and the ingrowth core comprises a bone scaffold structure (e.g., a resorbable polymer), so that the composite interference screw substantially completely disappears from the surgical site over time while yielding superior bone-to-ligament ingrowth.

The present invention also provides other novel apparatus for making attachments to bone. In general, the novel apparatus preferably comprises a frame for providing the short term strength needed to set the apparatus into position and to hold the various elements in position while bone ingrowth occurs, and an ingrowth core for promoting superior bone ingrowth. Preferably, the frame comprises a bioabsorbable polymer, and the ingrowth core comprises a bone scaffold structure (e.g., a resorbable polymer), so that the apparatus substantially completely disappears from the surgical site over time while yielding superior bone ingrowth. Among other things, this apparatus may comprise a spine cage for use in effecting spinal fusion, or an osteotomy wedge for use in effecting a high-tibial, open-wedge osteotomy.

In one form of the present invention, there is provided an interference screw comprising:

an open helical coil having a proximal end and a distal end aligned along a longitudinal axis and defining an internal volume, with the internal volume communicating with the region exterior to the open helical coil through the spacing between the turns of the open helical coil; and

at least one runner disposed within the internal volume and connected to multiple turns of the open helical coil.

In another form of the present invention, there is provided a method for attaching a graft ligament to a bone, the method comprising:

providing an interference screw comprising:
an open helical coil having a proximal end and a distal end aligned along a longitudinal axis and defining an internal volume, with the internal volume communicating with the region exterior to the open helical coil through the spacing between the turns of the open helical coil; and
at least one runner disposed within the internal volume and connected to multiple turns of the open helical coil;

forming a bone tunnel in the bone, and providing a graft ligament;

inserting the graft ligament into the bone tunnel; and

inserting the interference screw into the bone tunnel so as to secure the graft ligament to the bone.

In an additional form of the present invention, there is provided a bone cage comprising:

an open helical coil having a proximal end and a distal end aligned along a longitudinal axis and defining an internal volume, with the internal volume communicating with the region exterior to the open helical coil through the spacing between the turns of the open helical coil; and

at least one runner disposed within the internal volume and connected to multiple turns of the open helical coil.

In still another form of the present invention, there is provided a method for fusing together two portions of bone, the method comprising:

providing a bone cage comprising:
an open helical coil having a proximal end and a distal end aligned along a longitudinal axis and defining an internal volume, with the internal volume communicating with the region exterior to the open helical coil through the spacing between the turns of the open helical coil; and
at least one runner disposed within the internal volume and connected to multiple turns of the open helical coil;

forming a bone tunnel in the two portions of bone; and

inserting the bone cage into the bone tunnel so as to secure the two bone portions in position relative to one another while fusion occurs.

In yet another form of the present invention, there is provided a bone cage comprising:

a cage frame having a proximal end and a distal end aligned along a longitudinal axis and defining an internal volume, wherein the cage frame has a generally rectangular exterior geometry and the internal volume has a generally rectangular geometry, with the internal volume communicating with the region exterior to the cage frame through at least one window formed in the cage frame; and

an insert disposed within the internal volume;

wherein the cage frame is formed out of a material having substantial strength, and further wherein the insert is formed out of a bone scaffold material.

In still another form of the present invention, there is provided a method for fusing together two portions of bone, the method comprising:

providing a bone cage comprising:
a cage frame having a proximal end and a distal end aligned along a longitudinal axis and defining an internal volume, wherein the cage frame has a generally rectangular exterior geometry and the internal volume has a generally rectangular geometry, with the internal volume communicating with the region exterior to the cage frame through at least one window formed in the cage frame; and
an insert disposed within the internal volume;
wherein the cage frame is formed out of a material having substantial strength, and further wherein the insert is formed out of a bone scaffold material;

forming a bone tunnel in the two portions of bone; and

inserting the bone cage into the bone tunnel so as to secure the two bone portions in position relative to one another while fusion occurs.

In an additional form of the present invention, there is provided an osteotomy wedge comprising:

a wedge frame defining a wedge-shaped internal volume, with the internal volume communicating with the region exterior to the wedge frame through a plurality of apertures formed in the wedge frame; and

an insert disposed within the internal volume;

wherein the wedge frame is formed out of a material having substantial strength, and further wherein the insert is formed out of a bone scaffold material.

In still another form of the present invention, there is provided a method for performing an open wedge osteotomy, the method comprising:

providing an osteotomy wedge comprising:
a wedge frame defining a wedge-shaped internal volume, with the internal volume communicating with the region exterior to the wedge frame through a plurality of apertures formed in the wedge frame; and
an insert disposed within the internal volume;
wherein the wedge frame is formed out of a material having substantial strength, and further wherein the insert is formed out of a bone scaffold material;

forming a wedge-shaped opening in the bone; and

inserting the osteotomy wedge into the wedge-shaped opening in the bone.

### BRIEF DESCRIPTION OF THE DRAWINGS

These and other objects and features of the present invention will be more fully disclosed or rendered obvious by the following detailed description of the preferred embodiments of the invention, which is to be considered together with the accompanying drawings wherein like numbers refer to like parts, and further wherein:

FIG. 1 is a schematic side view showing a prior art interference screw securing a graft ligament to a bone;

FIG. 2 is a schematic top view of the construction shown in FIG. 1;

FIG. 3 is a side view of a novel composite interference screw formed in accordance with the present invention;

FIG. 4 is an exploded perspective view of the composite interference screw shown in FIG. 3;

FIG. 5 a perspective view of the composite interference screw shown in FIGS. 3 and 4, with selected portions of the screw frame being cut away;

FIGS. 6-13 illustrate one preferred method for effecting a ligament reconstruction utilizing the composite interference screw shown in FIGS. 3-5;

FIGS. 14-16 show a spinal fusion procedure using a conventional spine cage;

FIGS. 17-19 show a composite bone cage formed in accordance with the present invention;

FIGS. 20 and 21 show another composite bone cage formed in accordance with the present invention;

FIGS. 22-24 show a spinal fusion procedure using the composite bone cage of FIGS. 20 and 21;

FIGS. 25-27 show a high-tibial, open-wedge osteotomy;

FIGS. 28 and 29 show a composite osteotomy wedge formed in accordance with the present invention; and

FIGS. 30 and 31 show a high-tibial, open-wedge osteotomy procedure using the composite osteotomy wedge of FIGS. 28 and 29.

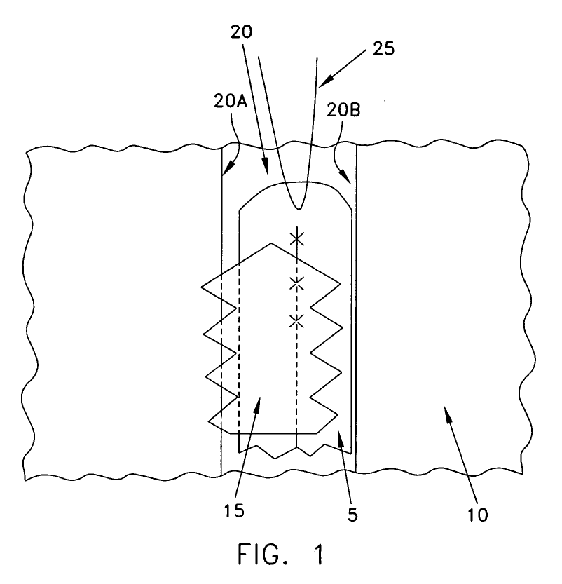

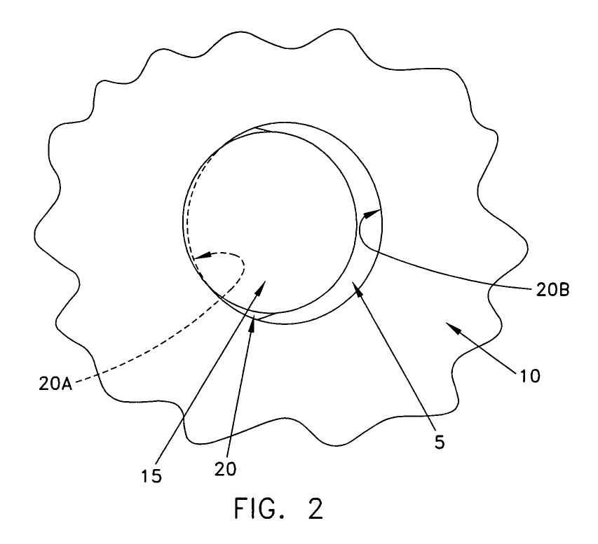

### DETAILED DESCRIPTION OF THE PREFERRED EMBODIMENTS

### Composite Interference Screw

The present invention provides a novel composite interfence screw for use in attaching a graft ligament to a bone. For convenience, the present invention will hereinafter be discussed in the context of its use for ACL tibial and/or femoral fixation; however, it should be appreciated that the present invention may also be used for the fixation of other graft ligaments to the tibia and/or the femur; and/or the fixation of other graft ligaments to other bones. Furthermore, the screw construction of the present invention may also be used to secure other objects (e.g., prosthetic devices, bone plates, etc.) to bone, and the screw construction of the present invention may also be used to secure bone to bone.

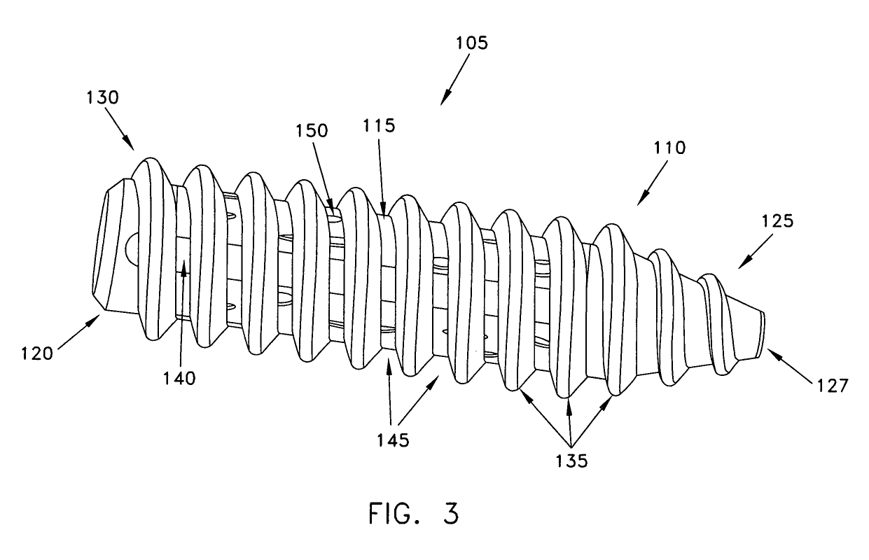

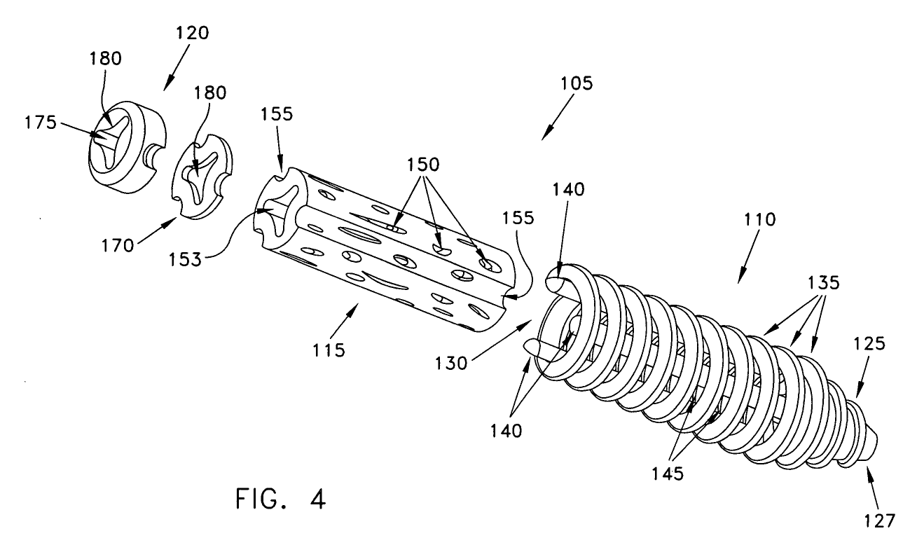

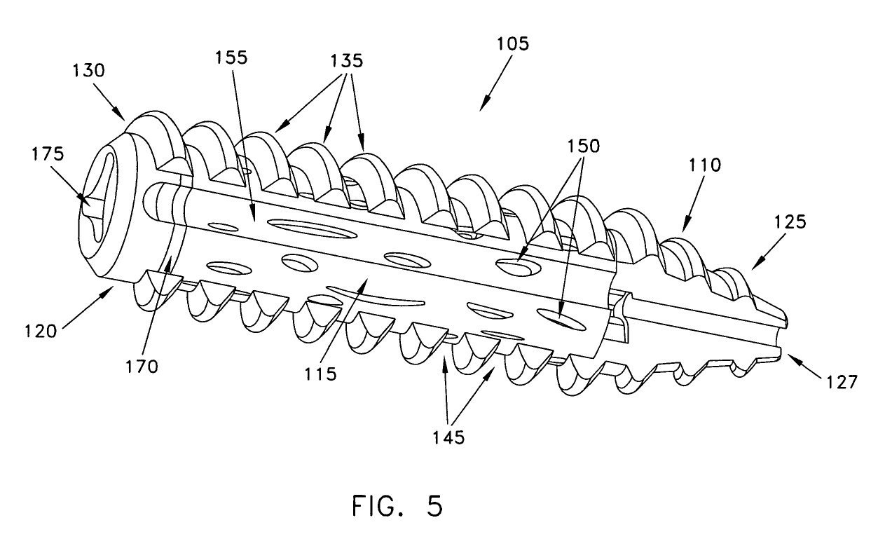

Looking next at FIGS. 3-5, there is shown a novel composite interference screw 105 for securing a graft ligament within a bone tunnel. Composite interference screw 105 generally comprises a screw frame 110 for providing the short term strength needed to set the interference screw into position and to hold the graft ligament in position while bone-to-ligament ingrowth occurs, an ingrowth core 115 for promoting superior bone-to-ligament ingrowth, and a cap 120 for closing off the proximal end of screw frame 110 and for use in advancing composite interference screw 105 into the bone tunnel.

Screw frame 110 comprises a distal end 125 and a proximal end 130. Distal end 125 is preferably generally conically-shaped, and preferably terminates in a narrowed tip 127 to allow for easy insertion of interference screw 105 into the bone tunnel. Screw frame 110 comprises screw threads 135 which extend in a helical fashion from distal end 125 to proximal end 130. If desired, screw frame 110 may also comprise a plurality of longitudinally-extending runners 140 extending along the interior of screw threads 135 from distal end 125 to proximal end 130.

Screw frame 110 comprises apertures 145 extending intermediate at least some of the screw threads 135. Apertures 145 facilitate contact between the side wall of the bone tunnel and ingrowth core 115, as will hereinafter be discussed. If desired, screw frame 110 may have a solid floor between all of the screw threads 135, and apertures 145 may comprise openings in the floor of screw frame 110. More preferably, however, screw threads 135 are in the form of a helicoil (i.e., an open helical coil), with apertures 145 being defined by the space between the turns of the coil, as shown in FIGS. 3-5. In other words, interference screw 105 may comprise an open helical coil defining an internal volume, with the internal volume communicating with the region exterior to the open helical coil through the spacing between the turns of the open helical coil.

Where screw threads 135 are in the form of a helicoil, runners 140 can help to provide support to the helicoil. Furthermore, where screw frame 110 is to be made with a molding process, runners 140 can be used to help flow the melt into position.

Preferably, the number of runners 140, and their size, are selected so as to close off an insignificant portion of the spacing between the turns of the helical coil, whereby to substantially not affect the communication of the internal volume with the region external to the open helical coil. At the same time, however, the number of runners 140, their size, and composition, are selected so as to provide any necessary support to the turns of the open helical coil.

In one preferred form of the present invention, one runner 140 is provided. In another preferred form of the present invention, a plurality of runners (e.g., two, three, four or more runners) are provided.

And in one preferred form of the present invention, the runners 140 collectively close off less than fifty percent of the spacing between the turns of the open helical coil.

And in one particularly preferred form of the present invention, the runners 140 collectively close off less than twenty percent of the spacing between the turns of the open helical coil.

Screw frame 110 is formed out of one or more biocompatible materials. These biocompatible materials may be non-absorbable (e.g., stainless steel or plastic) or absorbable (e.g., a bioabsorbable polymer). In one preferred form of the invention, screw frame 110 preferably comprises a bioabsorbable polymer such as polylactic acid (PLA), polyglycolic acid (PGA), etc. In any case, however, screw frame 110 comprises a material which is capable of providing the short term strength needed to set the interference screw into position and to hold the graft ligament in position while bone-to-ligament ingrowth occurs.

Ingrowth core 115 is disposed interior to screw frame 110 and is configured so as to promote superior bone ingrowth. Ingrowth core 115 preferably comprises a plurality of small perforations 150 which operate to increase the effective surface area of ingrowth core 115. Ingrowth core 115 may also comprise a central lumen 153 extending at least part way along the longitudinal axis of ingrowth core 115. In one preferred form of the invention, screw frame 110 is cannulated, and central lumen 153 is aligned with this cannulation, so that composite interference screw 105 can be deployed over a guidewire. By way of example but not limitation, distal end 125, and cap 120, of interference screw 105 may comprise axial openings for receiving a guidewire.

Ingrowth core 115 is configured to fit within screw frame 110. In one preferred form of the invention, where screw frame 110 comprises runners 140, ingrowth core 115 comprises corresponding longitudinal grooves 155 which complement runners 140, whereby to facilitate (i) insertion of ingrowth core 115 into screw frame 110, (ii) a “close fit” between ingrowth core 115 and screw frame 110, and (iii) a stabilized positioning of ingrowth core 115 relative to screw frame 110.

Ingrowth core 115 is formed out of one or more biocompatible materials which supports superior ligament-to-bone ingrowth. In one preferred form of the invention, ingrowth core 115 is formed out of a bone scaffold material or structure (e.g., a resorbable polymer) which provides a structure for new bone to grow on, with the structure thereafter slowly being replaced by bone, leaving only the new bone behind.

Preferably, ingrowth core 115 is formed using PolyGraft® material produced by OsteoBiologics, Inc. of San Antonio, Tex.

Alternatively, in another preferred form of the invention, ingrowth core 115 is formed using a different bone scaffold structure, e.g., a synthetic material, an allograft material (e.g., demineralized bone) and/or other material or materials (e.g., hydroxyapatite) which is substantially completely replaced by bone over time.

If desired, ingrowth core 115 may be doped with bone growth factors so as to enhance bone ingrowth.

Significantly, inasmuch as ingrowth core 115 is disposed within screw frame 110, ingrowth core 115 does not need to provide the short term strength needed to set the interference screw into position and to hold the graft ligament in position while bone-to-ligament ingrowth occurs, since this strength function is provided by screw frame 110. Thus, the material used to form ingrowth core 115 can be substantially optimized to provide the desired superior ingrowth characteristics, without regard to strength characteristics.

Cap 120 is attached to the proximal end 130 of screw frame 110 so as to (i) capture ingrowth core 115 within screw frame 110, and (ii) provide a means for turning interference screw 105, whereby to advance interference screw 105 into position within the bone tunnel. Preferably, cap 120 is overmolded onto the proximal end 130 of screw frame 110 so as to form a secure joinder. In this case, it may be desirable to provide a buffer 170 between cap 120 and ingrowth core 115 in order to protect ingrowth core 115 from the heat of molding. In one preferred form of the invention, screw frame 110, cap 120 and buffer 170 are all formed out of the same material (e.g., an absorbable polymer) and ingrowth core 115 is formed out of another material (e.g., a bone scaffold structure in the form of a resorbable polymer).

In one preferred form of the invention, cap 120 and buffer 170 are provided with central lumens 175 and 180, respectively, which are coaxial with lumen 153 of ingrowth core 115.

It is also possible to form composite interference screw 105 using an overmolding process. Thus, for example, in one form of the invention, screw frame 110 and cap 120 may be molded directly onto ingrowth core 115, assuming that the materials used to form ingrowth core 115 are not harmed by the molding conditions required to set screw frame 110 and cap 120 onto ingrowth core 115. In this case, buffer 170 might be omitted.

### Using the Composite Interference Screw to Attach a Graft Ligament to a Bone

Composite interference screw 105 may be employed in substantially the same manner as a conventional interference screw.

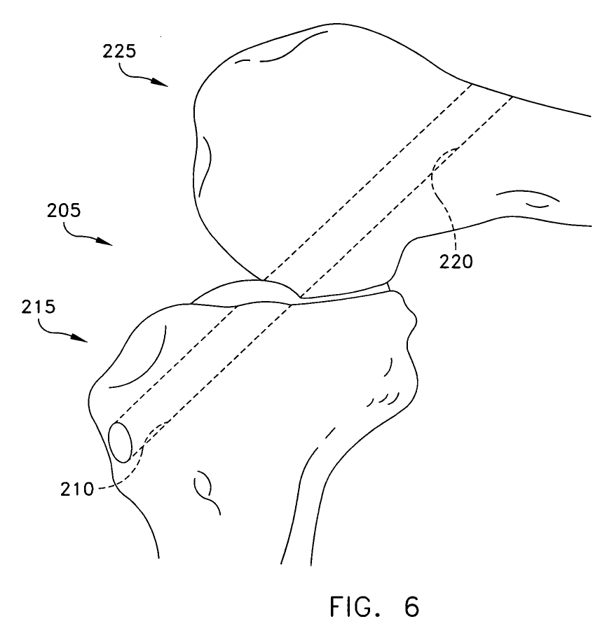

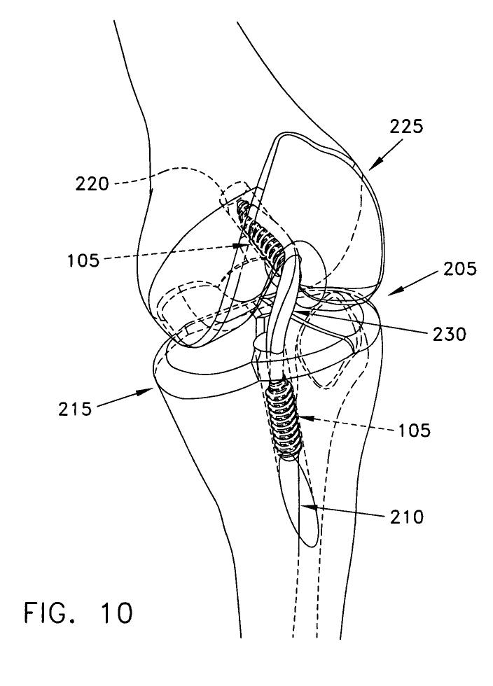

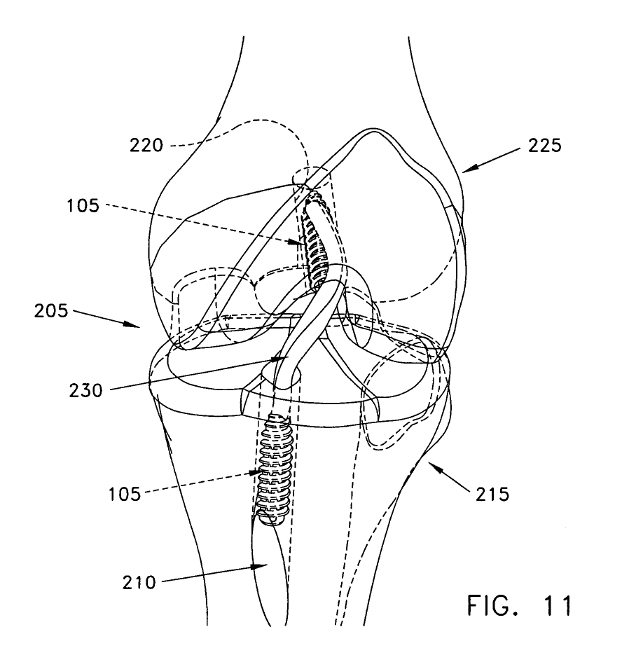

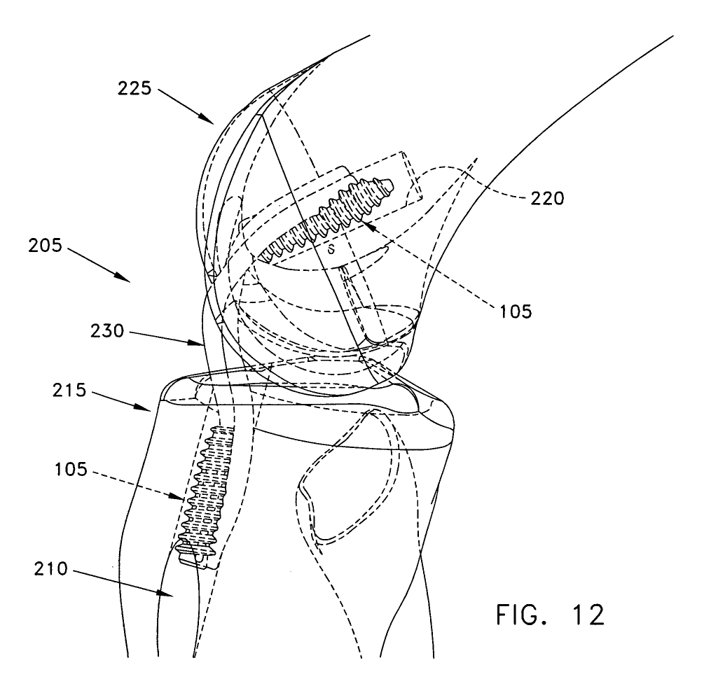

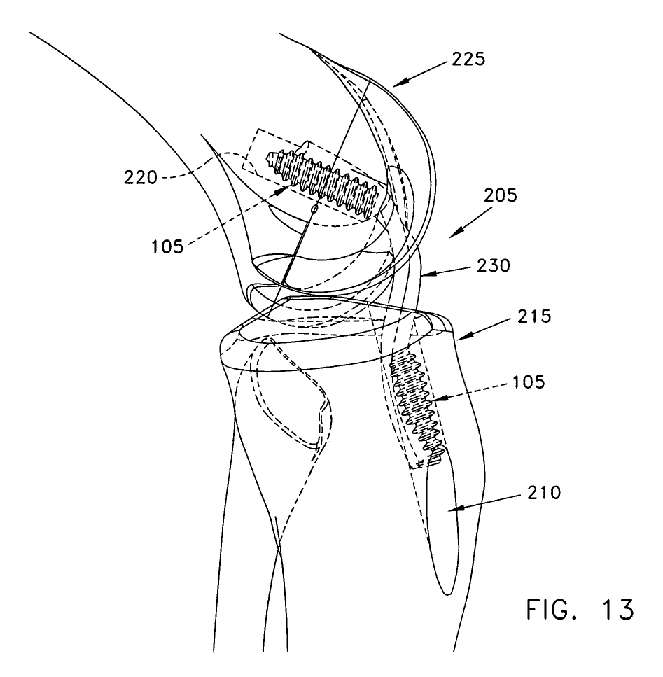

More particularly, and looking now at FIGS. 6-13, there are shown various aspects of an ACL reconstruction effected using composite interference screw 105.

FIG. 6 shows a typical knee joint 205, with the joint having been prepared for an ACL reconstruction, i.e., with the natural ACL having been removed, and with a tibial bone tunnel 210 having been formed in tibia 215, and with a femoral bone tunnel 220 having been formed in femur 225.

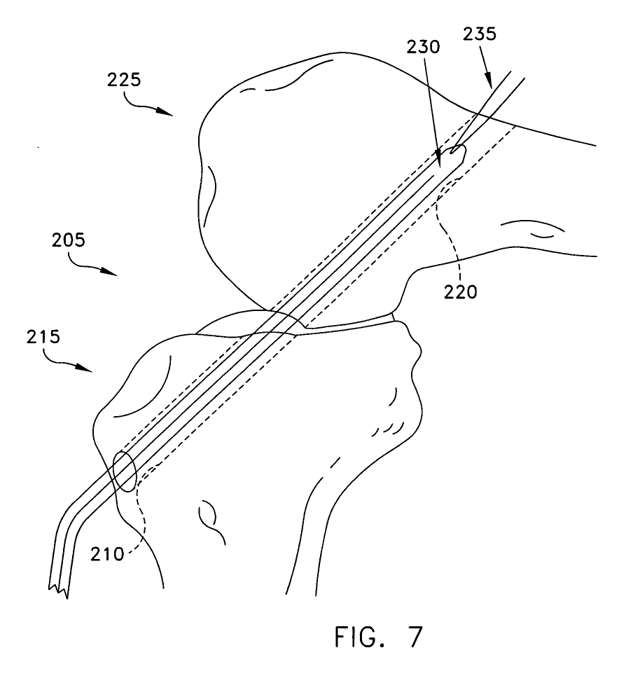

FIG. 7 is a view similar to that of FIG. 6, except that a graft ligament 230 has been positioned in femoral bone tunnel 220 and tibial bone tunnel 210 in accordance with ways well known in the art. By way of example, graft ligament 230 may be towed up through tibial bone tunnel 210 and into femoral bone tunnel 220 using a tow suture 235.

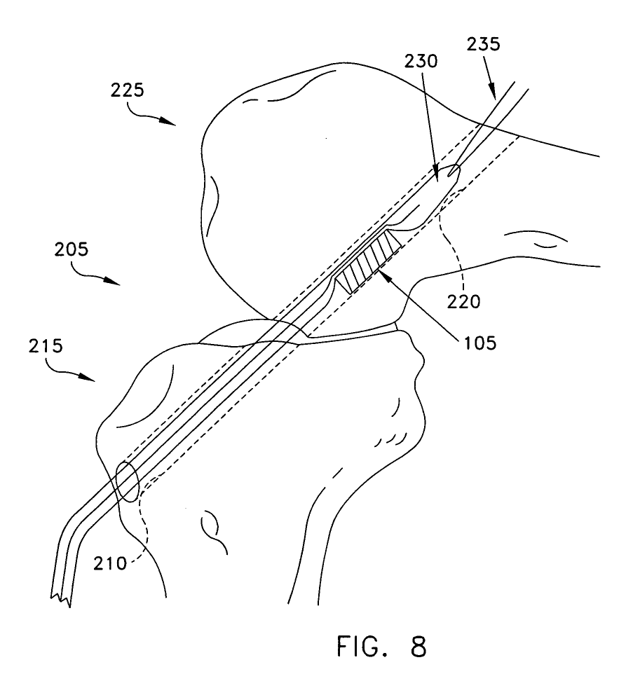

FIG. 8 shows graft ligament 230 made fast in femoral tunnel 220 using composite interference screw 105. More particularly, in accordance with the present invention, composite interference screw 105 may be mounted on an inserter (not shown) of the sort well known in the art, by fitting the distal tip of the inserter into central lumens 175 and 180, respectively, of cap 120 and buffer 170 and, to the extent desired, into central lumen 153 of ingrowth core 115. Furthermore, if desired, the inserter may be cannulated so that the inserter and interference screw may be deployed over a guidewire. Then the inserter is used to advance composite interference screw 105 up into the femoral tunnel, turning the inserter so as to rotationally drive composite interference screw 105, whereby to force composite interference screw 105 between the side wall of femoral bone tunnel 220 and graft ligament 230, thereby securing graft ligament 230 to the bone. As this occurs, screw frame 110 provides the short term strength needed to set the composite interference screw into position and to hold the graft ligament in position while bone-to-ligament ingrowth occurs. In this respect it will be appreciated that the superior ingrowth characteristics of ingrowth core 115 can provide the desired superior bone-to-ligament ingrowth. Furthermore, the apertures 145 in screw frame 110 provide the desired access to ingrowth core 115 even as screw frame 110 holds the graft ligament in position while bone-to-ligament ingrowth occurs. Over time, ingrowth core 115 is replaced with new bone, and screw frame 110 is absorbed by the body.

Significantly, forming screw frame 110 in the form of an open helical coil has proven particularly advantageous, inasmuch as the open helical coil provides the strength needed to set the interference screw into position and hold the graft ligament in position, while still providing extraordinary access to ingrowth core 115, whereby to facilitate superior bone ingrowth.

In addition, by virtue of the fact that ingrowth core 115 comprises longitudinal grooves 155 for receiving runners 140 of screw frame 110, ingrowth core 115 and screw frame 110 collectively provide a smooth outer profile, void of sharp edges, at the base of the helical coil. As a result, the smooth outer profile prevents interference screw 105 from cutting or tearing tissue (either hard tissue or soft tissue) as the interference screw is turned into tissue. This is in marked contrast to constructions where windows are provided in the floor of a screw thread. In these constructions, the edges of the windows provide sharp edges which can function like cutting flutes when the screw is turned into tissue.

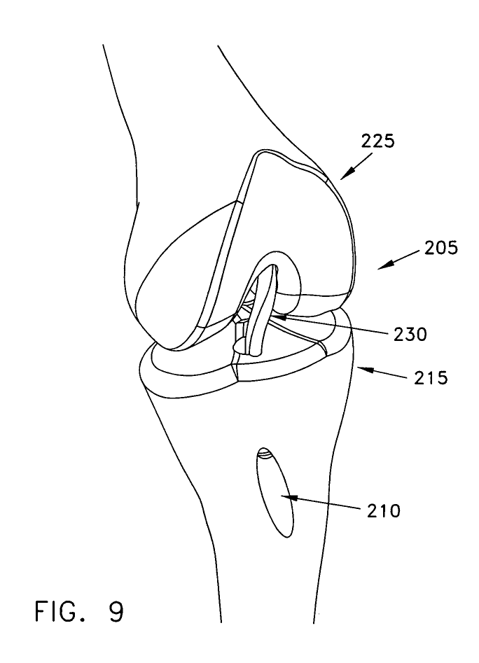

FIGS. 9-13 illustrate a complete ACL reconstruction using composite interference screws 105.

As noted above, forming screw frame 110 in the form of an open helical coil has proven particularly advantageous, since open helical coil provides the strength needed to set the interference screw into position and hold the graft ligament in position, while still providing extraordinary access to the region interior to the interference screw. In this respect, it should also be appreciated that the advantages of the open helical coil may be harnessed without using ingrowth core 115. More particularly, in this form of the present invention, a novel interference screw is provided which comprises an open helical coil without an internal ingrowth core 115. In this case, the open helical coil provides the strength needed to set the interference screw into position and hold the graft ligament in position, while still providing extraordinary access to the region interior to the interference screw. This arrangement has been found to provide excellent bone ingrowth results.

In this form of the present invention, when interference screw 105 is used without ingrowth core 115, the inserter for the interference screw is designed to fit within the interior volume of the open helical coil, with the inserter being provided with longitudinal grooves to receive runners 140 of screw frame 110. The engagement of the inserter with the runners allows the rotational motion of the inserter to be transferred to the interference screw, whereby to permit the inserter to rotationally drive the interference screw. Significantly, by virtue of the fact that the inserter comprises longitudinal grooves for receiving runners 140 of screw frame 110, the inserter and screw frame 110 collectively provide a smooth outer profile, void of sharp edges, at the base of the helical coil. As a result, the smooth outer profile prevents interference screw 105 from cutting or tearing tissue (either hard tissue or soft tissue) as the interference screw is turned into tissue. Again, this is in marked contrast to constructions where windows are provided in the floor of a screw thread. In these constructions, the edges of the windows provide sharp edges which can function like cutting flutes when the screw is turned into tissue.

### Bone Cages

It is also possible to use the present invention to create improved bone cages.

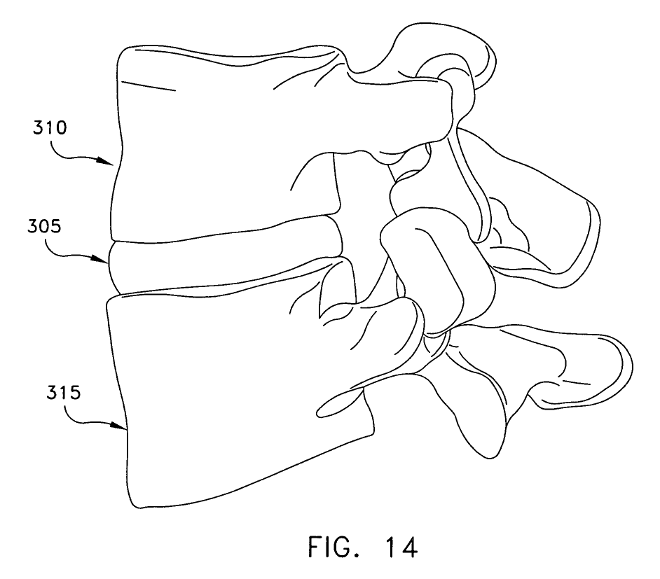

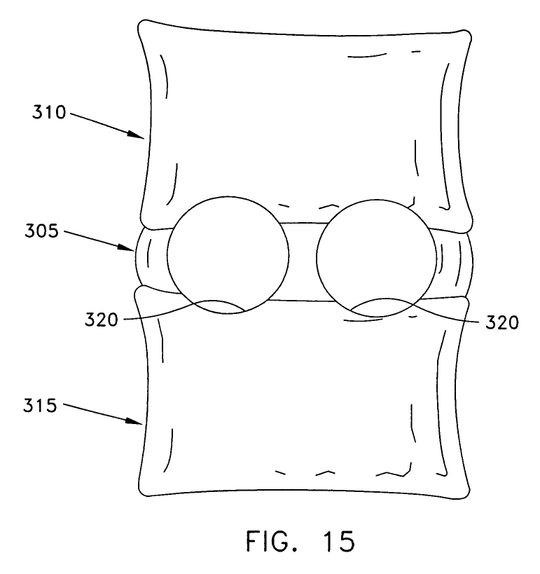

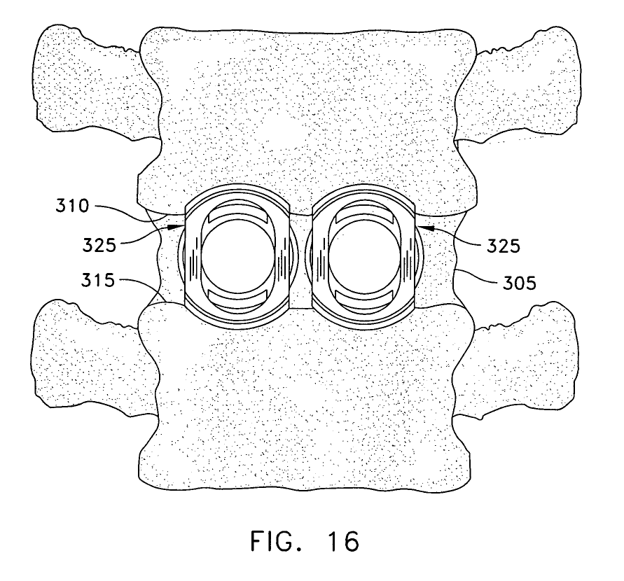

More particularly, bone cages may be used in bone fusion procedures to fuse together several portions of bone. In one common application, bone cages (sometimes referred to as spine cages) are used in a spinal fusion procedure where some or all of a diseased or damaged disc is removed and the two adjacent vertebrae fused together. FIG. 14 shows the natural patient anatomy, with a disc 305 sitting between two opposing vertebrae 310, 315 so as to support and cushion the vertebrae. When the disc is irreparably diseased or damaged, a spinal fusion may be performed. In this spinal fusion procedure, the disc is generally partially or fully removed and the two opposing vertebrae fused together. More particularly, in this procedure, a bone cage (or, more commonly, a pair of bone cages) are positioned between the two vertebrae so as to facilitate fusion of the two bones. This is typically done by first forming a cage seat 320 (FIG. 15) across the two vertebrae (e.g., by drilling and tapping), and then installing a bone cage 325 in the cage seat 320 (FIG. 16).

The present invention may be used to form an improved bone cage, i.e., a bone cage which (i) has the short term strength needed to set the apparatus into position and to hold the various elements in position while bone ingrowth occurs, (ii) promotes superior bone ingrowth, and (iii) substantially completely disappears from the surgical site over time.

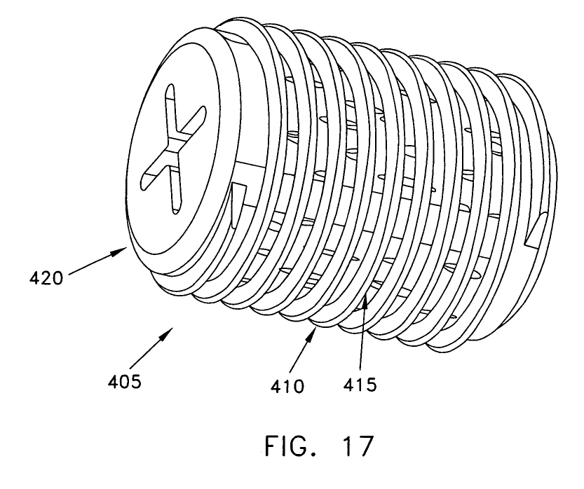

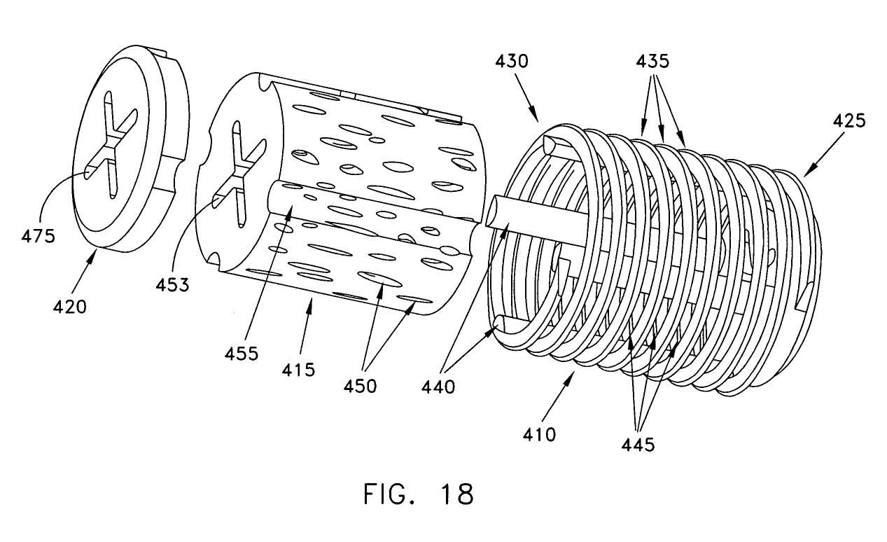

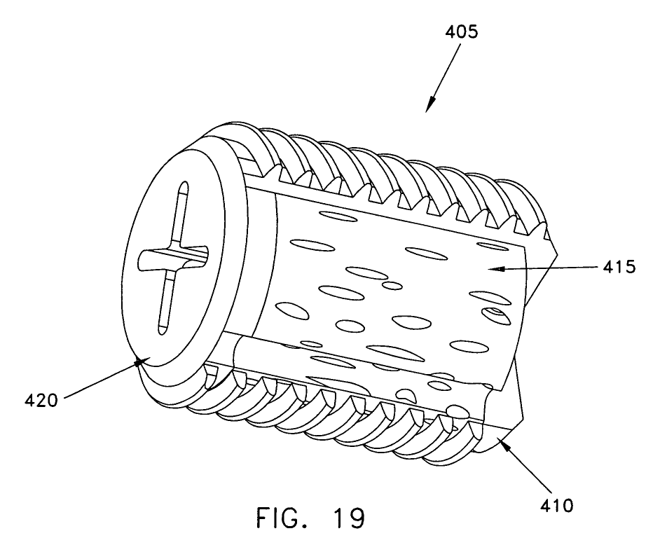

More particularly, FIGS. 17-19 show a composite bone cage 405 formed in accordance with the present invention. Composite bone cage 405 generally comprises a cage frame 410 for providing the short term strength needed to set the bone cage into position and to hold the bones in position while bone-to-bone ingrowth occurs, an ingrowth core 415 for promoting superior bone-to-bone ingrowth, and a cap 420 for closing off the proximal end of cage frame 410 and for use in advancing composite bone cage 405 into the cage seat.

Cage frame 410 comprises a distal end 425 and a proximal end 430. Cage frame 410 comprises screw threads 435 which extend in a helical fashion from distal end 425 to proximal end 430. If desired, cage frame 410 may also comprise a plurality of longitudinally-extending runners 440 extending along the interior of screw threads 435 from distal end 425 to proximal end 430.

Cage frame 410 comprises apertures 445 extending intermediate at least some of the screw threads 435. Apertures 445 facilitate contact between the side wall of the cage seat and ingrowth core 415, as will hereinafter be discussed. If desired, cage frame 410 may have a solid floor between all of the screw threads 435, and apertures 445 may comprise openings in the floor of screw frame 410. More preferably, however, screw threads 435 are in the form of a helicoil (i.e., an open helical coil), with apertures 445 being defined by the space between the turns of the coil, as shown in FIGS. 17-19. In other words, cage frame 410 may comprise an open helical coil defining an internal volume, with the internal volume communicating with the region exterior to the open helical coil through the spacing between the turns of the open helical coil.

Where screw threads 435 are in the form of a helicoil, runners 440 can help to provide support to the helicoil. Furthermore, where screw frame 110 is to be made with a molding process, runners 440 can be used to help flow the melt into position.

Preferably, the number of runners 440, and their size, are selected so as to close off an insignificant portion of the spacing between the turns of the helical coil, whereby to substantially not affect the communication of the internal volume with the region external to the open helical coil. At the same time, however, the number of runners 440, their size, and composition, are selected so as to provide any necessary support to the turns of the open helical coil.

In one preferred form of the present invention, one runner 440 is provided. In another preferred form of the present invention, a plurality of runners (e.g., two, three, four or more runners) are provided.

And in one preferred form of the present invention, the runners 440 collectively close off less than fifty percent of the spacing between the turns of the open helical coil.

And in one particularly preferred form of the present invention, the runners 440 collectively close off less than twenty percent of the spacing between the turns of the open helical coil.

Cage frame 410 is formed out of one or more biocompatible materials. These biocompatible materials may be non-absorbable (e.g., stainless steel or plastic) or absorbable (e.g., a bioabsorbable polymer). In one preferred form of the invention, cage frame 410 preferably comprises a bioabsorbable polymer such as polylactic acid (PLA), polyglycolic acid (PGA), etc. In any case, however, cage frame 410 comprises a material which is capable of providing the short term strength needed to set the bone cage into position and to hold the bones in position while bone-to-bone ingrowth occurs.

Ingrowth core 415 is disposed interior to cage frame 410 and is configured so as to promote superior bone ingrowth. Ingrowth core 415 preferably comprises a plurality of small perforations 450 which operate to increase the effective surface area of ingrowth core 415. Ingrowth core 415 may also comprise a central lumen 453 extending at least part way along the longitudinal axis of ingrowth core 415.

Ingrowth core 415 is configured to fit within cage frame 410. In one preferred form of the invention, where cage frame 410 comprises runners 440, ingrowth core 415 comprises corresponding longitudinal grooves 455 which complement runners 440, whereby to facilitate (i) insertion of ingrowth core 415 into cage frame 410, (ii) a “close fit” between ingrowth core 415 and cage frame 410, and (iii) a stabilized positioning of ingrowth core 415 relative to screw frame 110.

Ingrowth core 415 is formed out of one or more biocompatible materials which supports superior bone-to-bone ingrowth. In one preferred form of the invention, ingrowth core 415 is formed out of a bone scaffold material or structure (e.g., a resorbable polymer) which provides a structure for new bone to grow on, with the structure thereafter slowly being replaced by bone, leaving only the new bone behind.

Preferably, ingrowth core 415 is formed using PolyGraft® material produced by OsteoBiologics, Inc. of San Antonio, Tex.

Alternatively, in another preferred form of the invention, ingrowth core 415 is formed using a different bone scaffold structure, e.g., a synthetic material, an allograft material (e.g., demineralized bone) and/or other material or materials (e.g., hydroxyapatite) which is substantially completely replaced by bone over time.

If desired, ingrowth core 415 may be doped with bone growth factors so as to enhance bone ingrowth.

Significantly, inasmuch as ingrowth core 415 is disposed within cage frame 410, ingrowth core 415 does not need to provide the short term strength needed to set the bone cage into position and to hold the bones in position while bone-to-bone ingrowth occurs, since this strength function is provided by cage frame 410. Thus, the material used to form ingrowth core 415 can be substantially optimized to provide the desired superior ingrowth characteristics, without regard to strength characteristics.

Cap 420 is attached to the proximal end 430 of cage frame 410 so as to (i) capture ingrowth core 415 within cage frame 410, and (ii) provide a means for turning bone cage 405, whereby to advance bone cage 405 into position within the cage seat. Preferably, cap 420 is overmolded onto the proximal end 430 of cage frame 410 so as to form a secure joinder. In this case, it may be desirable to provide a buffer (not shown in FIGS. 17-19, but generally similar to the buffer 170 provided between cap 120 and ingrowth core 115 in the construction of composite interference screw 105) between cap 420 and ingrowth core 415 in order to protect ingrowth core 415 from the heat of molding. In one preferred form of the invention, cage frame 410 and cap 420 (and, if provided, the aforementioned buffer) are all formed out of the same material (e.g., an absorbable polymer) and ingrowth core 415 is formed out of another material (e.g., a bone scaffold structure in the form of a resorbable polymer).

In one preferred form of the invention, cap 420 (and, if provided, the buffer) is (are) provided with a central lumen 475 which is (are) coaxial with lumen 453 of ingrowth core 415.

It is also possible to form composite bone cage 405 using an overmolding process. Thus, for example, in one form of the invention, cage frame 410 and cap 420 may be molded directly onto ingrowth core 415, assuming that the materials used to form ingrowth core 415 are not harmed by the molding conditions required to set cage frame 410 and cap 420 onto ingrowth core 415.

Composite bone cage 405 is employed in substantially the same manner as a conventional bone cage. However, due to its unique construction, composite bone cage 405 provides superior performance. Specifically, cage frame 410 provides the short term strength needed to set the composite bone cage into position and to hold the opposing vertebrae in position while bone-to-bone ingrowth occurs. In this respect it will be appreciated that the superior ingrowth characteristics of ingrowth core 415 can provide the desired superior bone-to-bone ingrowth. Furthermore, the apertures 445 in cage frame 410 provide the desired access to ingrowth core 415 even as cage frame 410 holds the two vertebrae in position while bone-to-bone ingrowth occurs. Over time, ingrowth core 415 is replaced with new bone, and cage frame 410 is absorbed by the body.

Significantly, forming cage frame 410 in the form of an open helical coil has proven particularly advantageous, inasmuch as the open helical coil provides the strength needed to set the bone cage into position and hold the bones in position, while still providing extraordinary access to ingrowth core 415, whereby to facilitate superior bone ingrowth.

As noted above, forming cage frame 410 in the form of an open helical coil has proven particularly advantageous, since open helical coil provides the strength needed to set the bone cage into position and hold the bones in position, while still providing extraordinary access to the region interior to the bone cage. In this respect, it should also be appreciated that the advantages of the open helical coil may be harnessed without using ingrowth core 415. More particularly, in this form of the present invention, a novel bone cage is provided which comprises an open helical coil without an internal ingrowth core 415. In this case, the open helical coil provides the strength needed to set the bone cage into position and hold the bones in position, while still providing extraordinary access to the region interior to the bone cage. This arrangement has been found to provide excellent bone ingrowth results.

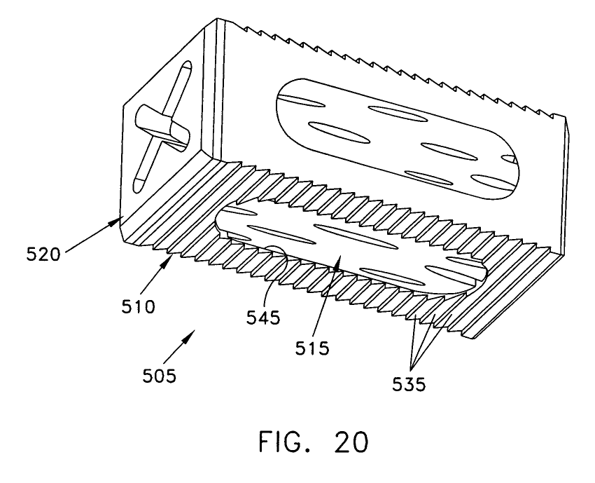

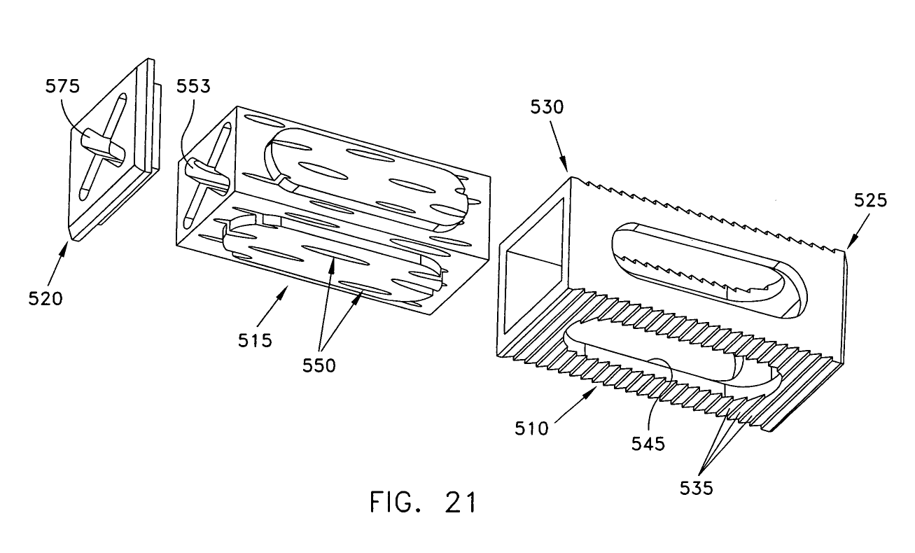

FIGS. 20 and 21 illustrate another composite bone cage 505 also formed in accordance with the present invention. Composite bone cage 505 is generally similar to the composite bone cage 405 discussed above, i.e., composite bone cage 505 comprises a cage frame 510 for providing the short term strength needed to set the bone cage into position and to hold the bones in position while bone-to-bone ingrowth occurs, an ingrowth core 515 for promoting superior bone-to-bone ingrowth, and a cap 520 for closing off the proximal end of cage frame 510 and for use in advancing composite bone cage 505 into a cage seat. However, cage frame 510, ingrowth core 515 and cap 520 are all formed with a generally rectangular cross-section (rather than the circular cross-section of composite bone cage 405), the screw threads 435 of cage frame 410 are replaced by ribs 535, and apertures 545 comprise elongated windows formed in the body of cage frame 510.

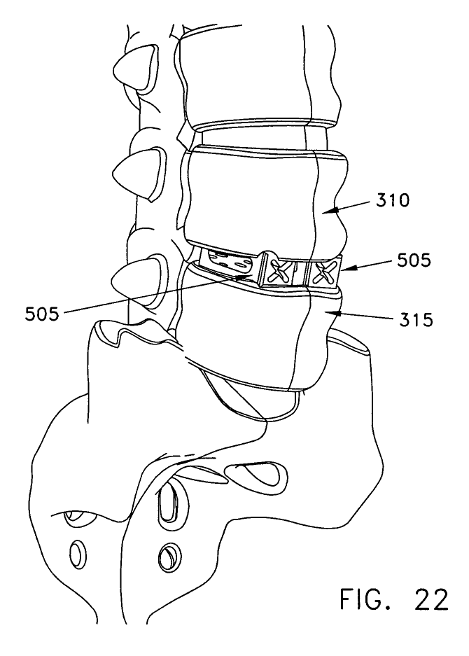

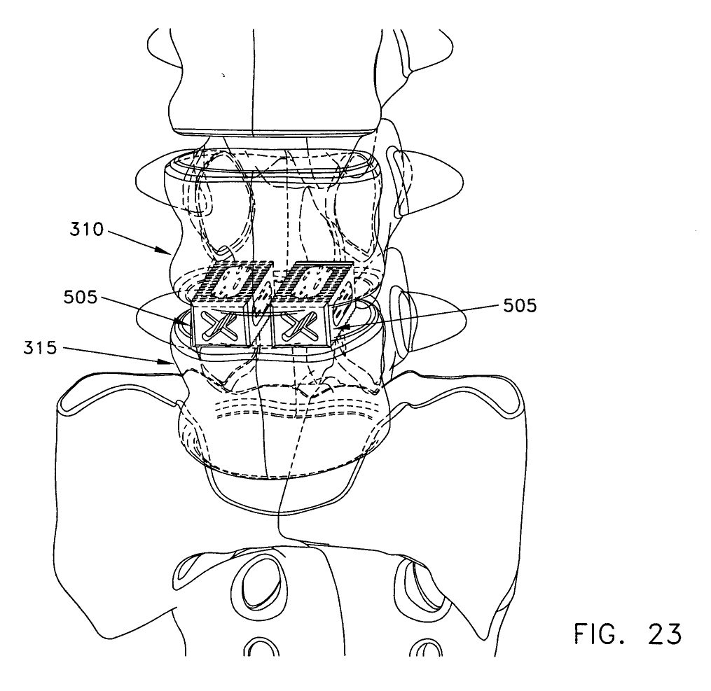

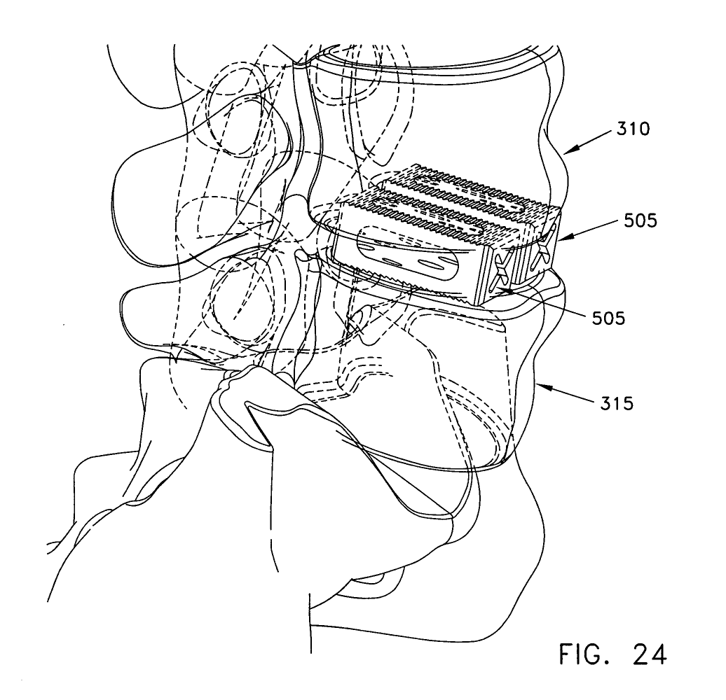

FIGS. 22-24 show a spinal fusion being effected using the composite bone cage 505. In this respect it will be appreciated that the cage seats formed in the patient's anatomy are also formed with a rectangular cross-section to match the rectangular cross-section of composite bone cages 505.

If desired, bone cage 505 may omit ingrowth core 515.

### Osteotomy Wedge

It is also possible to use the present invention to create an improved osteotomy wedge.

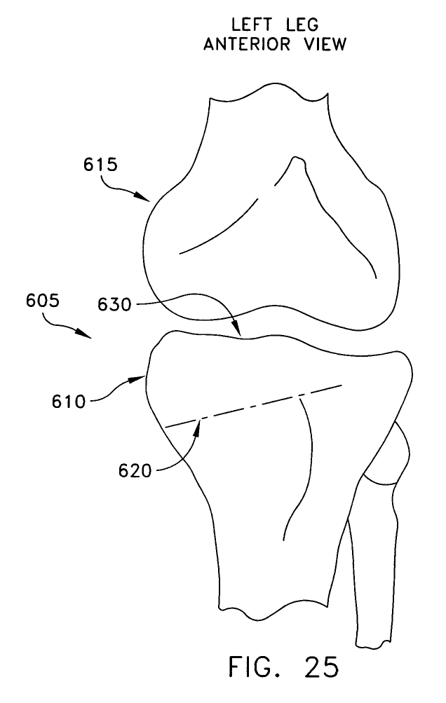

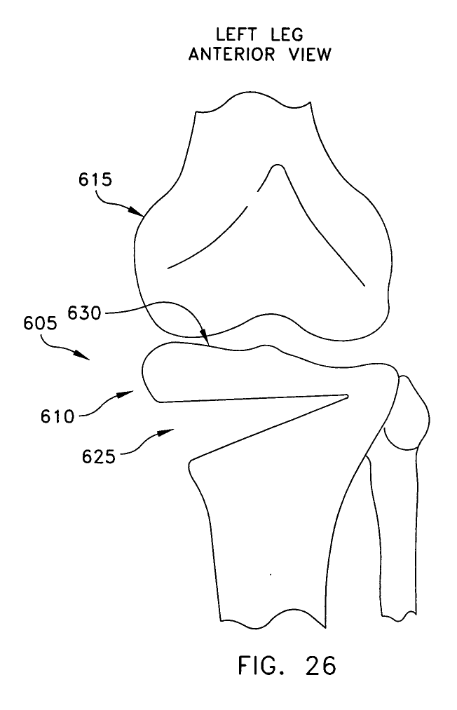

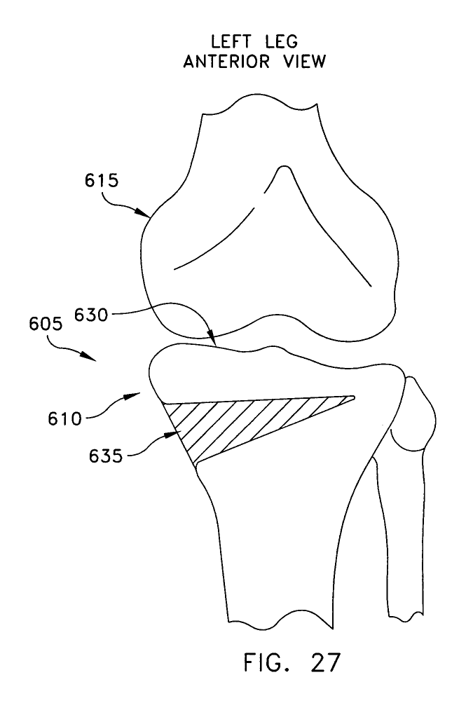

More particularly, an osteotomy wedge is typically used in a high-tibial, open-wedge osteotomy procedure where the top end of the tibia is reoriented so as to improve load transmission through the knee. FIG. 25 shows a knee joint 605 upon which an open wedge osteotomy is to be performed. Knee joint 605 generally comprises a tibia 610 and a femur 615. The high-tibial, open-wedge osteotomy is generally effected by first making a cut 620 into the upper tibia, and then moving apart the portions of the bone on either side of cut 620 so as to form a wedge-like opening 625 (FIG. 26) in the bone, with the wedge-like opening 625 being configured such that the tibial plateau 630 is given the desired orientation relative to femur 615. Once the desired wedge-like opening 625 has been formed in tibia 610 and tibial plateau 630 given its desired orientation, a wedge-shaped implant 635 (FIG. 27) is inserted into the wedge-like opening formed in the tibia so as to stabilize tibia 610 in its desired orientation.

The present invention may be used to form an improved osteotomy wedge, i.e., a composite osteotomy wedge which (i) has the short term strength needed to set the apparatus into position and to support the various elements in position while bone ingrowth occurs, (ii) promotes superior bone ingrowth, and (iii) substantially completely disappears from the surgical site over time.

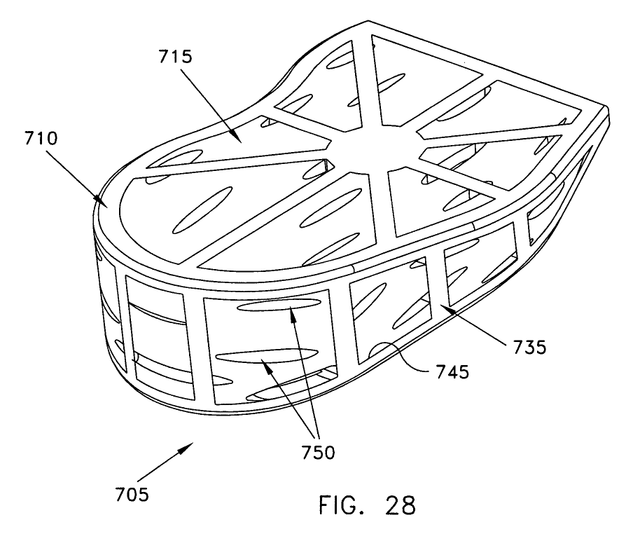

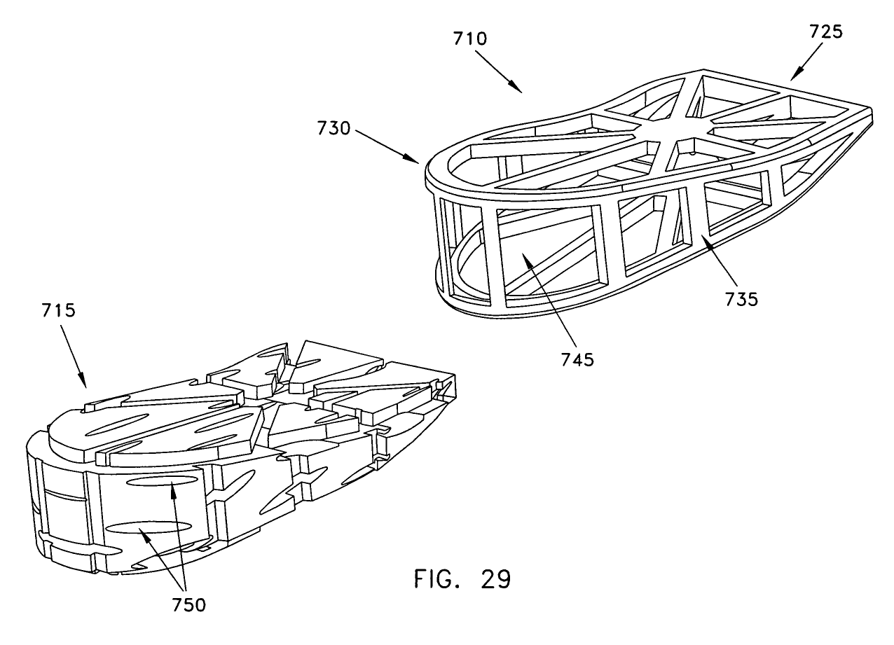

More particularly, FIGS. 28 and 29 show a composite osteotomy wedge 705 formed in accordance with the present invention. Composite osteotomy wedge 705 generally comprises a wedge frame 710 for providing the short term strength needed to set the osteotomy wedge into position and to support the bones in position while bone-to-bone ingrowth occurs, and an ingrowth core 715 for promoting superior bone-to-bone ingrowth.

Wedge frame 710 comprises a distal end 725 and a proximal end 730. Wedge frame 710 comprises a skeleton 735 which extends from distal end 725 to proximal end 730.

Wedge frame 710 comprises apertures 745 extending intermediate at least some of the skeleton. Apertures 745 facilitate contact between the cut surfaces of the tibia and ingrowth core 715, as will hereinafter be discussed. In one preferred form of the invention, wedge frame 710 comprises a multi-element skeleton 735 and apertures 745 comprise the spaces between the skeleton elements.

Wedge frame 710 is formed out of one or more biocompatible materials. These biocompatible materials may be non-absorbable (e.g., stainless steel or plastic) or absorbable (e.g., a bioabsorbable polymer). In one preferred form of the invention, wedge frame 710 preferably comprises a bioabsorbable polymer such as polylactic acid (PLA), polyglycolic acid (PGA), etc. In any case, however, wedge frame 710 comprises a material which is capable of providing the short term strength needed to set the osteotomy wedge into position and to support the bones in position while bone-to-bone ingrowth occurs.

Ingrowth core 715 is disposed interior to wedge frame 710 and is configured so as to promote superior bone ingrowth. Ingrowth core 715 preferably comprises a plurality of small perforations 750 which operate to increase the effective surface area of ingrowth core 715.

Ingrowth core 715 is configured to fit within wedge frame 710. In one preferred form of the invention, wedge frame 710 is molded over ingrowth core 715.

Ingrowth core 715 is formed out of one or more biocompatible materials which supports superior bone-to-bone ingrowth. In one preferred form of the invention, ingrowth core 715 is formed out of a bone scaffold structure (e.g., a resorbable polymer) which provides a structure for new bone to grow on, with the structure thereafter slowly being replaced by bone, leaving only the new bone behind.

Preferably, ingrowth core 715 is formed using PolyGraft® material produced by OsteoBiologics, Inc. of San Antonio, Tex.

Alternatively, in another preferred form of the invention, ingrowth core 715 is formed using a different bone scaffold material, e.g., a synthetic material, an allograft material (e.g., demineralized bone) and/or other material or materials (e.g., hydroxyapatite) which is substantially completely replaced by bone over time.

If desired, ingrowth core 715 may be doped with bone growth factors so as to enhance bone ingrowth.

Significantly, inasmuch as ingrowth core 715 is disposed within wedge frame 710, ingrowth core 715 does not need to provide the short term strength needed to set the composite osteotomy wedge into position and to support the bones in position while bone-to-bone ingrowth occurs, since this strength function is provided by wedge frame 710. Thus, the material used to form ingrowth core 715 can be substantially optimized to provide the desired superior ingrowth characteristics, without regard to strength characteristics.

Composite osteotomy wedge 705 is employed in substantially the same manner as a conventional osteotomy wedge. However, due to its unique construction, composite osteotomy wedge 705 provides superior performance. Specifically, wedge frame 710 provides the short term strength needed to set the composite osteotomy wedge into position and to stabilize the tibia while bone-to-bone ingrowth occurs. In this respect it will be appreciated that the superior ingrowth characteristics of ingrowth core 715 can provide the desired superior bone-to-bone ingrowth. Furthermore, the apertures 745 in wedge frame 710 will provide the desired access to ingrowth core 715 even as wedge frame 710 holds the two bone segments in position while bone-to-bone ingrowth occurs. Over time, ingrowth core 715 is replaced with new bone, and wedge frame 710 is absorbed by the body.

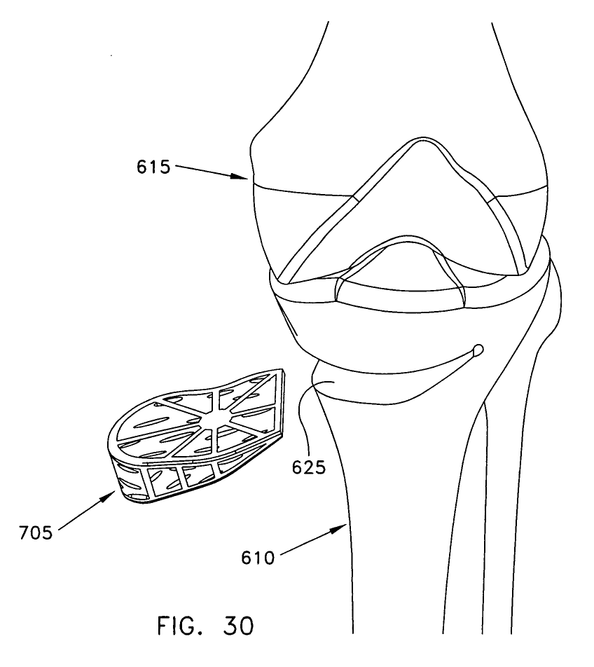

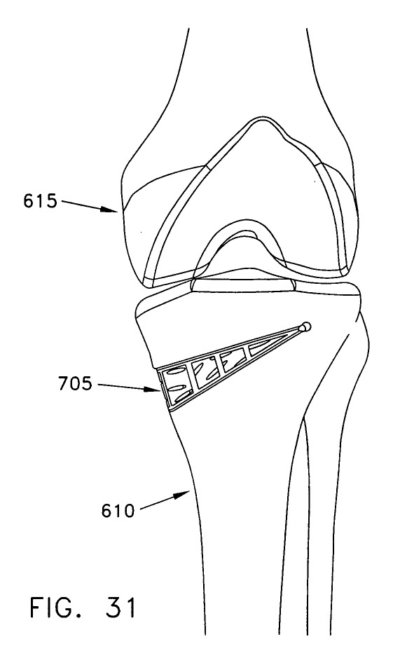

FIGS. 30 and 31 show a high-tibial, open-wedge osteotomy being effected using the composite osteotomy wedge 705.

If desired, osteotomy wedge 705 may omit ingrowth core 715.

### MODIFICATIONS

It will be appreciated that still further embodiments of the present invention will be apparent to those skilled in the art in view of the present disclosure. It is to be understood that the present invention is by no means limited to the particular constructions and method steps herein disclosed and/or shown in the drawings, but also comprises any modifications or equivalents within the scope of the invention.

What is claimed is:

1. An interference screw comprising:
an open helical coil having a proximal end and a distal end aligned along a longitudinal axis and defining an internal volume, with the internal volume communicating with the region exterior to the open helical coil through the spacing between the turns of the open helical coil; and
at least one runner disposed within the internal volume and connected to multiple turns of the open helical coil.

2. An interference screw according to claim 1 wherein the interference screw further comprises a nose disposed at the distal end of the open helical coil.

3. An interference screw according to claim 2 wherein the nose is tapered.

4. An interference screw according to claim 2 wherein the nose is formed integral with the open helical coil.

5. An interference screw according to claim 4 wherein the nose is formed as a solid body having a bore formed therein, wherein the bore is aligned with the longitudinal axis of the open helical coil.

6. An interference screw according to claim 5 wherein the nose includes surface threads which form a continuation of the open helical coil, with the space between the surface threads of the nose being filled by the solid body of the nose.

7. An interference screw according to claim 2 wherein the interference screw further comprises a cap disposed at the proximal end of the open helical coil.

8. An interference screw according to claim 7 wherein the cap is formed separately from the open helical coil, and further wherein the cap is secured to the open helical coil during a subsequent manufacturing step.

9. An interference screw according to claim 1 wherein the at least one runner is aligned with the longitudinal axis of the open helical coil.

10. An interference screw according to claim 1 wherein there are two runners.

11. An interference screw according to claim 1 wherein there are three runners.

12. An interference screw according to claim 1 wherein the at least one runner is sized so as to close off an insignificant portion of the spacing between the turns of the open helical coil, whereby to substantially not affect the communication of the internal volume with the region exterior to the open helical coil.

13. An interference screw according to claim 1 wherein the at least one runner is sized so as to close off less than fifty percent of the spacing between the turns of the open helical coil.

14. An interference screw according to claim 1 wherein the at least one runner is sized so as to close off less than twenty percent of the spacing between the turns of the open helical coil.

15. An interference screw according to claim 1 wherein the interference screw comprises a plurality of runners.

16. An interference screw according to claim 15 wherein the plurality of runners are sized so as to collectively close off an insignificant portion of the spacing between the turns of the open helical coil, whereby to substantially not affect the communication of the internal volume with the region exterior to the open helical coil.

17. An interference screw according to claim 15 wherein the plurality of runners are sized so as to collectively close off less than fifty percent of the spacing between the turns of the open helical coil.

18. An interference screw according to claim 15 wherein the plurality of runners are sized so as to collectively close off less than twenty percent of the spacing between the turns of the open helical coil.

19. An interference screw according to claim 1 wherein the open helical coil is made out of a non-absorbable material.

20. An interference screw according to claim 1 wherein the open helical coil is made out of an absorbable material.

21. An interference screw according to claim 1 wherein the interference screw further comprises a nose disposed at the distal end of the open helical coil, and further wherein the open helical coil and the nose are formed out of the same material.

22. An interference screw according to claim 1 wherein the interference screw further comprises a cap disposed at the proximal end of the open helical coil, and further wherein the open helical coil and the cap are formed out of the same material.

23. An interference screw according to claim 1 wherein the interference screw further comprises an insert disposed within the internal volume.

24. An interference screw according to claim 23 wherein the insert is formed out of a bone scaffold material.

25. An interference screw according to claim 24 wherein the bone scaffold material comprises a resorbable polymer.

26. An interference screw according to claim 23 wherein the insert has an external geometry which matches the internal cross-sectional geometry of the interference screw.

27. An interference screw according to claim 23 wherein the open helical coil comprises a generally cylindrical internal volume, wherein the insert comprises a generally cylindrical external geometry, and further wherein the insert includes at least one groove for receiving the at least one runner.

28. An interference screw according to claim 23 wherein the insert has at least one perforation.

29. An interference screw according to claim 23 wherein the open helical coil and the insert are formed out of different materials.

30. An interference screw according to claim 23 wherein the insert is formed separately from the open helical coil, and further wherein the insert is positioned within the internal volume during a subsequent manufacturing step.

31. An interference screw according to claim 30 wherein the interference screw further comprises a cap disposed at the proximal end of the open helical coil, and further wherein the cap is secured to the open helical coil after the insert is positioned within the internal volume.

32. A method for attaching a graft ligament to a bone, the method comprising:
providing an interference screw comprising:
an open helical coil having a proximal end and a distal end aligned along a longitudinal axis and defining an internal volume, with the internal volume communicating with the region exterior to the open helical coil through the spacing between the turns of the open helical coil; and
at least one runner disposed within the internal volume and connected to multiple turns of the open helical coil;
forming a bone tunnel in the bone, and providing a graft ligament;
inserting the graft ligament into the bone tunnel; and
inserting the interference screw into the bone tunnel so as to secure the graft ligament to the bone.

33. A bone cage comprising:
an open helical coil having a proximal end and a distal end aligned along a longitudinal axis and defining an internal volume, with the internal volume communicating with the region exterior to the open helical coil through the spacing between the turns of the open helical coil; and
at least one runner disposed within the internal volume and connected to multiple turns of the open helical coil.

34. A bone cage according to claim 33 wherein the bone cage further comprises a cap disposed at the proximal end of the open helical coil.

35. A bone cage according to claim 34 wherein the cap is formed separately from the open helical coil, and further wherein the cap is secured to the open helical coil during a subsequent manufacturing step.

36. A bone cage according to claim 33 wherein the at least one runner is sized so as to close off an insignificant portion of the spacing between the turns of the open helical coil, whereby to substantially not affect the communication of the internal volume with the region exterior to the open helical coil.

37. A bone cage according to claim 33 wherein the at least one runner is sized so as to close off less than fifty percent of the spacing between the turns of the open helical coil.

38. A bone cage according to claim 33 wherein the at least one runner is sized so as to close off less than twenty percent of the spacing between the turns of the open helical coil.

39. A bone cage according to claim 33 wherein the bone cage comprises a plurality of runners.

40. A bone cage according to claim 39 wherein the plurality of runners are sized so as to collectively close off an insignificant portion of the spacing between the turns of the open helical coil, whereby to substantially not affect the communication of the internal volume with the region exterior to the open helical coil.

41. A bone cage according to claim 39 wherein the plurality of runners are sized so as to collectively close off less than fifty percent of the spacing between the turns of the open helical coil.

42. A bone cage according to claim 39 wherein the plurality of runners are sized so as to collectively close off less than twenty percent of the spacing between the turns of the open helical coil.

43. A bone cage according to claim 33 wherein the bone cage further comprises a cap disposed at the proximal end of the open helical coil, and further wherein the open helical coil and the cap are formed out of the same material.

44. A bone cage according to claim 33 wherein the bone cage further comprises an insert disposed within the internal volume.

45. A bone cage according to claim 44 wherein the insert is formed out of a bone scaffold material.

46. A bone cage according to claim 45 wherein the bone scaffold material comprises a resorbable polymer.

47. A bone cage according to claim 44 wherein the insert has an external geometry which matches the internal cross-sectional geometry of the bone cage.

48. A bone cage according to claim 44 wherein the open helical coil and the insert are formed out of different materials.

49. A bone cage according to claim 48 wherein the helical coil is formed out of a material having substantial strength, and further wherein the insert is formed out of a bone scaffold material.

50. A method for fusing together two portions of bone, the method comprising:
providing a bone cage comprising:
an open helical coil having a proximal end and a distal end aligned along a longitudinal axis and defining an internal volume, with the internal volume communicating with the region exterior to the open helical coil through the spacing between the turns of the open helical coil; and
at least one runner disposed within the internal volume and connected to multiple turns of the open helical coil;
forming a bone tunnel in the two portions of bone; and
inserting the bone cage into the bone tunnel so as to secure the two bone portions in position relative to one another while fusion occurs.

51. A bone cage comprising:
a cage frame having a proximal end and a distal end aligned along a longitudinal axis and defining an internal volume, wherein the cage frame has a generally rectangular exterior geometry and the internal volume has a generally rectangular geometry, with the internal volume communicating with the region exterior to the cage frame through at least one window formed in the cage frame; and
an insert disposed within the internal volume;
wherein the cage frame is formed out of a material having substantial strength, and further wherein the insert is formed out of a bone scaffold material.

52. A bone cage according to claim 51 wherein the bone cage further comprises a cap disposed at the proximal end of the cage frame, and further wherein the cap is secured to the cage frame after the insert is positioned within the internal volume.

53. A method for fusing together two portions of bone, the method comprising:
providing a bone cage comprising:
a cage frame having a proximal end and a distal end aligned along a longitudinal axis and defining an internal volume, wherein the cage frame has a generally rectangular exterior geometry and the internal volume has a generally rectangular geometry, with the internal volume communicating with the region exterior to the cage frame through at least one window formed in the cage frame; and
an insert disposed within the internal volume;
wherein the cage frame is formed out of a material having substantial strength, and further wherein the insert is formed out of a bone scaffold material;
forming a bone tunnel in the two portions of bone; and
inserting the bone cage into the bone tunnel so as to secure the two bone portions in position relative to one another while fusion occurs.

54. An osteotomy wedge comprising:
a wedge frame defining a wedge-shaped internal volume, with the internal volume communicating with the region exterior to the wedge frame through a plurality of apertures formed in the wedge frame; and
an insert disposed within the internal volume;
wherein the wedge frame is formed out of a material having substantial strength, and further wherein the insert is formed out of a bone scaffold material.

55. A method for performing an open wedge osteotomy, the method comprising:
providing an osteotomy wedge comprising:
a wedge frame defining a wedge-shaped internal volume, with the internal volume communicating with the region exterior to the wedge frame through a plurality of apertures formed in the wedge frame; and
an insert disposed within the internal volume;
wherein the wedge frame is formed out of a material having substantial strength, and further wherein the insert is formed out of a bone scaffold material;
forming a wedge-shaped opening in the bone; and
inserting the osteotomy wedge into the wedge-shaped opening in the bone.
